(19) United States
(12) Patent Application Publication
(10) Pub. No.: US 2026/0130676 A1
(43) Pub. Date: May 14, 2026

(54) APPARATUS AND METHOD FOR FORMING A CAVITY IN A SACROILIAC JOINT

(71) Applicant: VGI Medical, LLC, Largo, FL (US)

(72) Inventors: Steven Anagnost, Largo, FL (US); Tov Inge Vestgaarden, Madeira Beach, FL (US)

(21) Appl. No.: 19/386,429

(22) Filed: Nov. 12, 2025

Related U.S. Application Data
(60) Provisional application No. 63/719,630, filed on Nov. 12, 2024.

(51) Int. Cl. A61B 17/16; A61B 17/17; A61B 17/56

(52) U.S. Cl. CPC A61B 17/1671; A61B 17/1757; A61B 2017/564

(57) ABSTRACT

Apparatus for forming a seat for receiving an implant, the apparatus comprising: a directional cannula having a distal end, a proximal end, and a cannula lumen extending therebetween; a punch tool guide having a distal end, a proximal end, and a punch tool guide lumen extending therebetween; a hole punch comprising a distal end and a proximal end; wherein the cannula lumen is sized to slidably receive the punch tool guide therein, and the guide lumen is sized to slidably receive the hole punch therein; wherein when the punch tool guide is disposed in the cannula lumen such that the distal end of the punch tool guide is disposed at a distalmost position, the distal end of the punch tool guide extends distally of the distal end of the directional cannula, and further wherein when the hole punch is disposed in the punch tool guide lumen such that the distal end of the hole punch is disposed at a distalmost position, the distal end of the hole punch extends distally of the distal end of the punch tool guide.

### REFERENCE TO PENDING PRIOR PATENT APPLICATIONS

This patent application claims benefit of pending prior U.S. Provisional Patent Application Ser. No. 63/719,630, filed Nov. 12, 2024 by VGI Medical, LLC and Steven Anagnost et al. for PUNCH TOOL FOR FORMING A CAVITY IN THE SACROILIAC JOINT (Attorney's Docket No. VG-12 PROV).

The above-identified patent application is hereby incorporated herein by reference.

### FIELD OF THE INVENTION

This invention relates to surgical instruments and methods in general, and more particularly to novel tools and methods for introducing an implant into a sacroiliac joint.

### BACKGROUND OF THE INVENTION

Lower back pain is a common ailment which often results in pain and suffering as well as loss of work time. Effective treatments for lower back pain will alleviate considerable patient suffering and provide economic benefits by reducing employee absenteeism.

In the past, lower back pain and leg pain have been attributed to herniated discs or other injuries to the spinal column. However, it has been recognized that extensive therapy and treatment is often unsuccessful in alleviating such pain. More recently, it has been recognized that at least some forms of lower back and leg pain can be attributed to symptomatic sacroiliac joint dysfunction or instability of the sacroiliac joint.

The sacroiliac joint is located at the juncture of the ilium (i.e., the upper bone of the pelvis, commonly called the hip bone), and the sacrum (i.e., located at the base of the lumbar spine, where it connects with the L5 vertebra). The function of the sacroiliac joint is the transmission of forces from the spine to the lower extremities and vice-versa. The sacroiliac joint is supported by a range of ligaments, including the sacroiliac ligament at the base of the joint and the anterior sacroiliac ligament at the top of the joint.

The sacroiliac joint has a limited range of motion. Nutation, the relative movement between the sacrum and ilium, is typically only about one to two degrees. Despite the limited range of motion between the sacrum and the ilium, a patient's sacroiliac joint can become damaged resulting in hypermobility of the joint. Hypermobility of the sacroiliac joint is very difficult to diagnose due to the small range of motion. Therefore, lower back pain or leg pain caused by sacroiliac joint dysfunction (e.g., degenerative sacroiliitis, inflammatory sacroiliitis, iatrogenic instability of the sacroiliac joint, osteitis condensans ilii, traumatic fracture dislocation of the pelvis, etc.) often goes misdiagnosed or undiagnosed.

In patients where sacroiliac joint pain is unresponsive to non-operative treatments, e.g. medication, physical therapy, chiropractic care and steroid injections, surgical stabilization is prescribed. Joint fusion is a surgical treatment that may be employed in order to relieve pain generated from sacroiliac joint dysfunction.

Placing an implant (sometimes also referred to herein as a “fusion implant”) into the sacroiliac joint space is challenging because the ilium protrudes and blocks easy access to the sacroiliac joint. Part of the ilium can be cut and removed in order to improve access, however, such cutting weakens the ilium, extends the time required for surgery and recovery from surgery, and increases the patient's pain.

In addition, there can exist a significant amount of soft tissue between the patient's skin and the sacroiliac joint. Surgeons generally wish to minimize soft tissue trauma to the patient, even when accessing a joint that is a significant distance away from the patient's skin. Since fusion of the sacroiliac joint generally requires delivering an implant into the sacroiliac joint so as to span the joint space, and since such implants tend to be extremely small while also requiring delivery into the joint in a particular prescribed orientation, significant retraction of tissue is generally needed in order to fully expose the sacroiliac joint.

Ideally such a joint fusion procedure can be performed in a minimally-invasive manner by utilizing a cannula to deliver the implant (and such other surgical tools as are required for the procedure) to the joint space. However, the aforementioned protruding ilium creates an obstacle that must be accommodated by such a cannula.

FIGS. 1A-8D depict an illustrative embodiment of a prior art implant delivery system and method for effecting fusion of a sacroiliac joint 10.

The prior art method comprises the steps of taking anterior, posterior, and lateral X-ray views of the sacroiliac (SI) area to identify the anatomy that is causing pain in a patient.

An entry point is established to gain access to the SI joint with an oblique (approximately thirty five degree (35°) angle) and a Ferguson angle measurement of curvature which is approximately ten to fifteen degrees (10-15°).

Looking first at FIG. 1A, while using an imaging device (e.g., X-ray, fluoroscopy) to image the SI joint, a first guide wire 12 is placed on top of the skin over the SI joint. Then, as depicted in FIG. 1B, the patient's skin is marked with a first straight line 14 to indicate the position of first guide wire 12 over the SI joint. First straight line 14 is drawn through SI joint 10 from the superior position of the joint to the inferior position of the joint.

Looking next at FIG. 2A, while using an imaging device (e.g., X-ray, fluoroscopy) to image the SI joint, a second guide wire 16 is placed on top of the skin over the superior section of joint 10 in intersecting relation to first guide wire 12. Then, as depicted in FIG. 2B, the patient's skin is marked with a second straight line 18 to indicate the position of second guide wire 16.

Looking next at FIG. 3A, while using an imaging device (e.g., X-ray, fluoroscopy) to image the SI joint, a third guide wire 20 is placed on top of the skin over the inferior section of joint 10 in intersecting relation to first guide wire 12. Then, as depicted in FIG. 3B, the patient's skin is marked with a third straight line 22 to indicate the position of third guide wire 20.

Three incision points 24, 26 and 28 are then marked on the skin, as depicted in FIG. 4.

Center incision point 24 is midway between the points where the second and third lines 18 and 22 cross first line 14 and is spaced in the medial direction about three to five millimeters (3-5 mm) from first line 14.

Superior incision point 26 is spaced in the medial direction about three to five millimeters (3-5 mm) from the intersection of first line 14 and second line 18.

Inferior incision point 28 is spaced in the medial direction about three to five millimeters (3-5 mm) from the intersection of first line 14 and third line 22.

As depicted in FIG. 5, a fourth guide wire 30 is inserted through center incision point 24, with a superior/inferior angle perpendicular to the patient. The medial/lateral angle is the same as the oblique angle on the C-arm, which is approximately thirty-five degrees (35°). Fourth guide wire 30 is guided into the SI joint and an incision is made when guide wire 30 is properly positioned.

A joint locator 50, depicted in FIGS. 6A-C, is then placed over guide wire 30, i.e., in ensleeving relation to said guide wire 30. A surface of joint locater 50 is etched black and that black-etched surface is positioned so that it faces the ilium.

A directional cannula 52, depicted in FIGS. 7A-7G, is then placed over joint locator 50, i.e., in ensleeving relation to said joint locator. A surface of directional cannula 52 is etched black and that black-etched surface matches the black-etched surface of joint locator 50, i.e., the black-etched surface of directional cannula 52 is also positioned so that it faces the ilium.

Directional cannula 52 has a cylindrical main body 52a having longitudinally-extending central bore 52b formed therein. Central bore 52b has a transverse profile that matches the transverse profile of a fusion implant. A leading end 52g of central bore 52b is exposed to view by cut-out or notch 52c. Two prongs 52d and 52e extend from said leading end in transversely spaced, parallel relation to one another. Prongs 52d and 52e enter the sacroiliac joint (i.e., the space between the sacrum and the ilium) when directional cannula 52 is in use. Barb 52f is formed in the leading end of main body 52a as depicted in FIGS. 7B-7E. Barb 52f engages the sacrum to prevent slippage when prongs 52d and 52e enter into sacroiliac joint 10.

FIG. 7G depicts implant 58 that is captured within bore 52b. Implant 58 is exposed to view because it is in the region of bore 52b where notch 52c is formed. Since the radial depth of notch 52c is less than half the diameter of bore 52b, implant 58 cannot fall from bore/lumen 52b. In other words, as indicated in FIG. 7H, notch 52c creates a “C”-shaped bore where the two (2) spaced apart points of the “C” are closer together than the widest part of implant 58, thereby retaining the implant within bore 52b.

Joint locator 50 and guide wire 30 are then retracted, leaving directional cannula 52 in position. When so positioned, prongs 52d, 52e are disposed in sacroiliac joint 10. Barb 52f engages the sacrum to hold directional cannula 52 in position as aforesaid with notch 52c accommodating the ilium.

A drill guide 54 is then inserted into the central bore or lumen of directional cannula 52 towards the sacrum to verify placement of directional cannula 52 in the SI joint. The lumen of directional cannula 52 has a profile that enables it to slidingly receive drill guide 54 in the center of the larger implant-receiving lumen.

Drill guide 54, depicted in FIGS. 8A-8D, has longitudinally extending eccentric bores 54a, 54b formed therein. Head 54c is enlarged relative to main body 54d that is rectangular in transverse section. Step 54e is formed where the transverse extent of main body 54d is reduced. Drill bit-accommodating semi-circular grooves 54 f and 54g are the continuation of bores 54a, 54b and are formed in opposite sides of the leading end of drill guide 54, said leading end being the part of main body 54d that extends distal of said step 54e.

A drill bit 56, depicted in FIG. 9 having positive stop 56a, is then inserted into eccentric bore 54a and said drill bit 56 is slid towards the sacrum until it abuts the sacrum.

The distance from the proximal end of head 54c of drill guide 54 to the lower side of positive stop 56a is then measured. Directional cannula 52 is properly seated in the SI joint if the measured distance is between twenty-five to thirty millimeters (25-30 mm). The proper seating can also be confirmed with a lateral X-ray view that shows the leading end of directional cannula 52 disposed flush with the sacrum.

Drill bit 56 is then inserted into eccentric drill guide bore 54a and a first cavity is created in the ilium by a first drilling, until positive stop 56a abuts the proximal end of directional cannula 52. Drill bit 56 is then withdrawn from bore 54a, placed into bore 54b, and a second cavity is formed in the ilium by a second drilling. The second drilling continues until drill bit 56 reaches positive stop 56a.

Drill guide 54 is then retracted from directional cannula 52 and rotated one hundred eighty degrees (180°). Drill bit 56 is then inserted into eccentric drill guide bore 54a and a first cavity is created in the sacrum by a third drilling that continues until drill bit 56 reaches positive stop 56a. Drill bit 56 is then withdrawn from bore 54a, placed into bore 54b, and a second cavity is formed in the sacrum by a fourth drilling. The fourth drilling continues until drill bit 56 reaches positive stop 56a. Drill bit 56 is then removed.

Due to the eccentricity of the bores and the rotation of the drill guide, all four cavities merge into a single cavity that accommodates the fusion implant.

When the drilling is completed, drill guide 54 is retracted from directional cannula 52 and fusion implant 58, depicted in FIG. 10, is inserted into the lumen of directional cannula 52. The chamfer is inserted downward.

Fusion implant 58 may take many forms and may be as simple as a dowel having a circular cross-section, i.e., the oval shape of main body 58a, upper and lower fins 58b, 58c, and the swept back leading edge 58d of said fins are not critical parts of the fusion implant.

An implant tamp 60, depicted in FIG. 11, includes head 60a and flat, elongate main body 60b. A positive stop, not numbered, is formed where main body 60b meets head 60a. Main body 60b is inserted into the lumen of directional cannula 52 to advance the implant. Head 60a is repeatedly tamped lightly with a hammer or other suitable tool, not illustrated, until the aforesaid positive stop abuts directional cannula 52. This should fully seat implant 58 in the SI joint, i.e., implant 58 should be countersunk into the SI joint by a distance of about three to five millimeters (3-5 mm). A lateral X-ray view is taken to confirm full deployment of implant 58.

As perhaps best understood in connection with FIGS. 7B and 7C, implant 58 emerges from bore 52b at cut-out or notch 52c. Notch 52c has an extent or depth less than half the diameter of cylindrical main body 52a as depicted in FIGS. 7C and 7E. Implant 58 is thus supported from below by the part of main body 52a that is not removed to form notch 52c. The dimensions of implant 58 allow it to be inserted into bore 52b, but prevent it from falling out upon exiting bore 52b, i.e., upon exposure to or open communication with notch 52c, i.e., implant 58 is supported from below by the longitudinally-extending part of notch 52c as perhaps best understood in connection with FIG. 7C. Implant 58 is inside the cavity drilled into the sacrum and ilium when said implant extends beyond leading end 52g of cylindrical main body 52 a.

The following procedure is then followed to insert additional implants 58 into cavities formed in the sacrum and the ilium.

A guide wire is inserted through superior incision point 26 at a superior/interior angle about forty-five degrees (45°) towards superior. The medial/lateral angle is the same as the oblique angle on the C-arm, which is approximately thirty-five degrees (35°).

The guide wire is then guided into the SI joint. A second incision is made when the guide wire is properly positioned. The steps that follow the first incision are then repeated, i.e., joint locator 50 is inserted over the guide wire, directional cannula 52 is paced over the joint locator, and so on.

The procedure is concluded by inserting a guide wire through inferior incision point 28 at a superior/interior angle about forty-five degrees (45°) towards inferior. The medial/lateral angle is the same as the oblique angle on the C-arm, which is approximately thirty-five degrees (35°).

The guide wire is then guided into the SI joint. A third incision is made when the guide wire is properly positioned. The steps that follow the first and second incisions are then repeated, i.e., joint locator 50 is inserted over the guide wire, directional cannula 52 is paced over the joint locator, and so on.

FIGS. 12A-K provide a simplified overview of the procedure. FIG. 12A is a front elevational diagrammatic representation of a sacrum 70 flanked by a pair of iliums 72a, 72b, showing two (2) sacroiliac joints 74a, 74b and FIG. 12B is a first perspective view of the diagrammatic representation of FIG. 12A, indicating how iliums 72a, 72b protrude forwardly relative to sacrum 70. FIG. 12C is a second perspective view of the diagrammatic representation of FIG. 12A and FIG. 12D is a view like FIG. 12C but adding notched cannula 52, showing how notch 52c accommodates a protruding ilium. FIG. 12E depicts the same parts as FIG. 12D but taken from a left perspective and FIG. 12F is the same view as FIG. 12E but including drill bit 56. FIG. 12G is the same as FIG. 12F but taken from a right perspective. FIG. 12H depicts cavity 76 after drilling is complete and the cannula and drill bit have been withdrawn. FIG. 12I is a like FIG. 12H but from a different perspective, FIG. 12J depicts implant 58 in drilled cavity 76, and FIG. 12K depicts the same structure as FIG. 12J but from a different perspective.

While the foregoing system and method for effecting fusion of a sacroiliac joint is effective, it is increasingly desirable to reduce the total number of surgical tools that must be employed for such a procedure in order to reduce the length and/or cost of the procedure. Furthermore, while osteotomes for cutting bone are known in the prior art and soft tissue biopsy devices are known in the prior art, such osteotomes and soft tissue biopsy devices are not configured for forming a seat in bone that is to receive an implant, let alone for forming a seat in two bones adjacent to an articulating joint simultaneously.

Thus there is a need for novel method and apparatus for delivering an implant to the sacroiliac joint which accommodates the surrounding anatomy and reduces the total number of surgical tools needed for the joint fusion procedure, whereby to allow for correcting symptomatic sacroiliac joint dysfunction or instability, enhancing stability for purposes of immobilizing a joint, and fusing two opposed bone structures across the joint.

### SUMMARY OF THE INVENTION

The present invention comprises novel methods and apparatus for delivering an implant to the sacroiliac joint which accommodates the surrounding anatomy and reduces the total number of surgical tools needed for the joint fusion procedure, whereby to allow for correcting symptomatic sacroiliac joint dysfunction or instability, enhancing stability for purposes of immobilizing a joint, and fusing two opposed bone structures across the joint.

In one preferred form of the invention, there is provided apparatus for forming a seat for receiving an implant, the apparatus comprising:
a directional cannula having a distal end, a proximal end, and a cannula lumen extending therebetween;
a punch tool guide having a distal end, a proximal end, and a punch tool guide lumen extending therebetween;
a hole punch comprising a distal end and a proximal end;
wherein the cannula lumen is sized to slidably receive the punch tool guide therein, and the guide lumen is sized to slidably receive the hole punch therein;
wherein when the punch tool guide is disposed in the cannula lumen such that the distal end of the punch tool guide is disposed at a distalmost position, the distal end of the punch tool guide extends distally of the distal end of the directional cannula, and further wherein when the hole punch is disposed in the punch tool guide lumen such that the distal end of the hole punch is disposed at a distalmost position, the distal end of the hole punch extends distally of the distal end of the punch tool guide.

In another preferred form of the invention, there is provided a method for forming a cavity in tissue for receiving an implant, the method comprising:
providing apparatus for forming a cavity for receiving an implant, the apparatus comprising:
   a directional cannula having a distal end, a proximal end, and a cannula lumen extending therebetween;
   a punch tool guide having a distal end, a proximal end, and a punch tool guide lumen extending therebetween; and
   a hole punch comprising a distal end and a proximal end;
disposing the distal end of the directional cannula against two bones of a joint;
inserting the punch tool guide into the cannula lumen of the directional cannula and advancing the distal end of the punch tool guide distally towards the joint such that the distal end of the punch tool guide extends distal to the distal end of the directional cannula;
inserting the hole punch into the punch tool guide lumen of the punch tool guide and advancing the distal end of the hole punch distally towards the joint such that the distal end of the hole punch extends distal to the distal end of the directional cannula and the distal end of the punch tool guide; and
forming a cavity in the two bones of the joint.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1A is an X-ray view depicting the first guide wire placed on the skin (i.e., external to the sacroiliac joint) to identify the location of the sacroiliac joint;

FIG. 1B is a diagrammatic view of a mark made on a patient's skin to indicate the position of the guide wire used to locate the sacroiliac joint in FIG. 1A;

FIG. 2A is an X-ray view depicting the second guide wire placed on the skin (i.e., external to the sacroiliac joint) to identify the superior portion of the sacroiliac joint;

FIG. 2B is a diagrammatic view of a mark made on a patient's skin to indicate the position of the guide wire used to identify the superior portion of the sacroiliac joint in FIG. 2A;

FIG. 3A is an X-ray view depicting the third guide wire placed on the skin (i.e., external to the sacroiliac joint) to identify the inferior portion of the sacroiliac joint;

FIG. 3B is a diagrammatic view of a mark made on a patient's skin to indicate the position of the guide wire used to identify the inferior portion of the sacroiliac joint in FIG. 3A;

FIG. 4 is a diagrammatic view of three incision points that are marked on the patient's skin after the markings in FIGS. 1B, 2B, and 3B have been made;

FIG. 5 is an X-ray view depicting a guide wire inserted through the central incision point of the three incision points of FIG. 4;

FIG. 6A is a perspective view of a joint locator;

FIG. 6B is a side elevational view of the joint locator;

FIG. 6C is a top plan view of the joint locator;

FIG. 7A is a perspective view of a novel directional cannula;

FIG. 7B is a top plan view of the directional cannula of FIG. 7A;

FIG. 7C is a side elevational view of the directional cannula of FIG. 7A;

FIG. 7D is a bottom plan view of the directional cannula of FIG. 7A;

FIG. 7E is an end view of the leading end of the directional cannula of FIG. 7A;

FIG. 7F is an end view of the trailing end of the directional cannula of FIG. 7A;

FIG. 7G is a side elevational view similar to FIG. 7C, but depicting an implant exposed to view by a notch but captured within a lumen so that it cannot fall;

FIG. 7H is an end view taken along lines 7H-7H in FIG. 7G;

FIG. 8A is a perspective view of a novel drill guide;

FIG. 8B is a side elevational view of the drill guide of FIG. 8A;

FIG. 8C is an end elevational view of the leading end of the drill guide of FIG. 8A;

FIG. 8D is an end elevational view of the trailing end of the drill guide of FIG. 8A;

FIG. 9 is a perspective view of a drill bit;

FIG. 10 is a perspective view of a fusion implant device;

FIG. 11 is a perspective view of a tamp;

FIG. 12A is a front elevational diagrammatic representation of a sacrum flanked by a pair of iliums, showing two (2) sacroiliac joints;

FIG. 12B is a first perspective view of the diagrammatic representation of FIG. 12A, indicating how the iliums protrude forwardly relative to the sacrum;

FIG. 12C is a second perspective view of the diagrammatic representation of FIG. 12A;

FIG. 12D is a view like FIG. 12C but adding a notched cannula, showing how the notch accommodates a protruding ilium;

FIG. 12E depicts the same parts as FIG. 12D but taken from a left perspective;

FIG. 12F is the same view as FIG. 12E but including the drill bit;

FIG. 12G is the same as FIG. 12F but taken from a right perspective;

FIG. 12H depicts the cavity after drilling is complete and the cannula and drill bit have been withdrawn;

FIG. 12I is a like FIG. 12H but from a different perspective;

FIG. 12J depicts an implant in the drilled cavity;

FIG. 12K depicts the same structure as FIG. 12J but from a different perspective; and

FIGS. 13-24 are schematic views showing a novel punch tool for forming a cavity in the sacroiliac joint formed in accordance with the present invention.

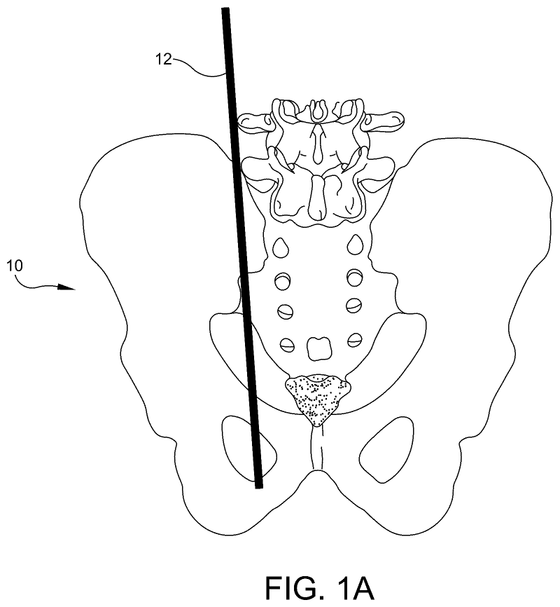

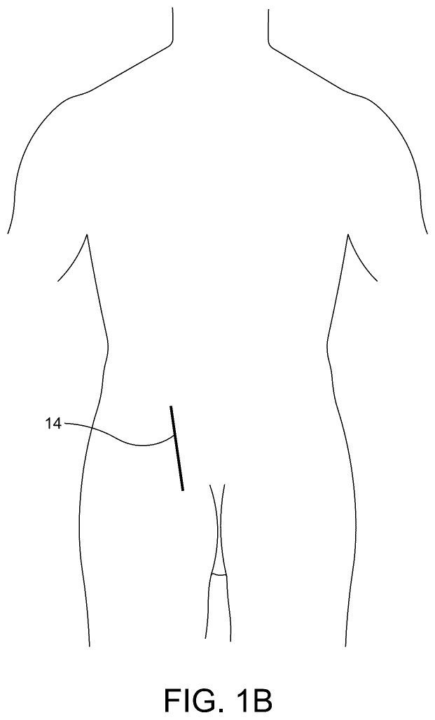

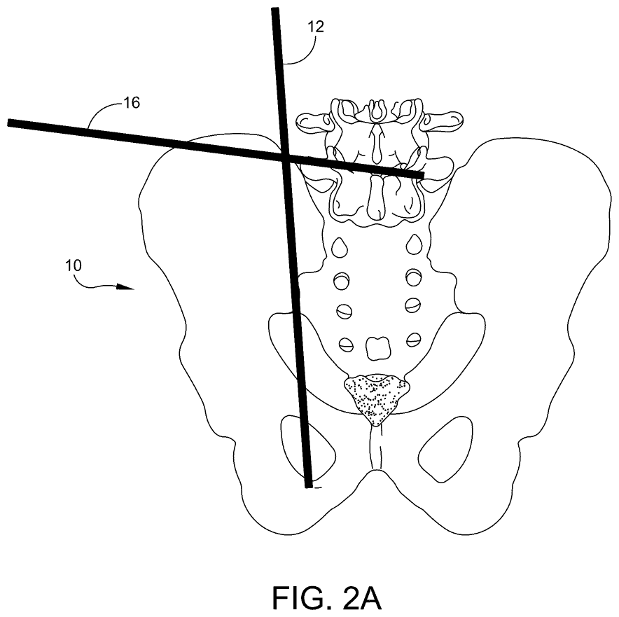

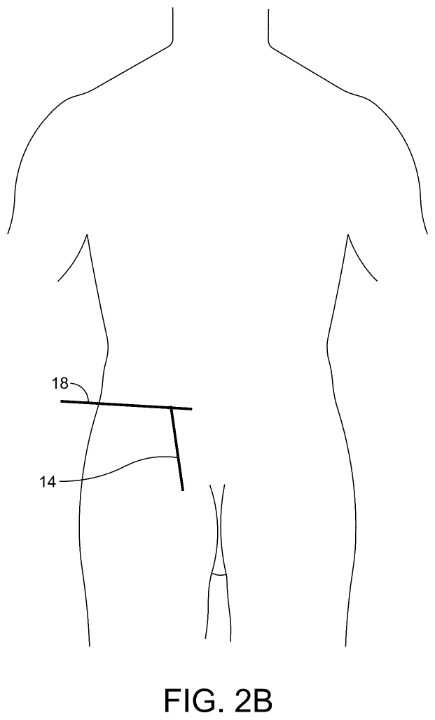

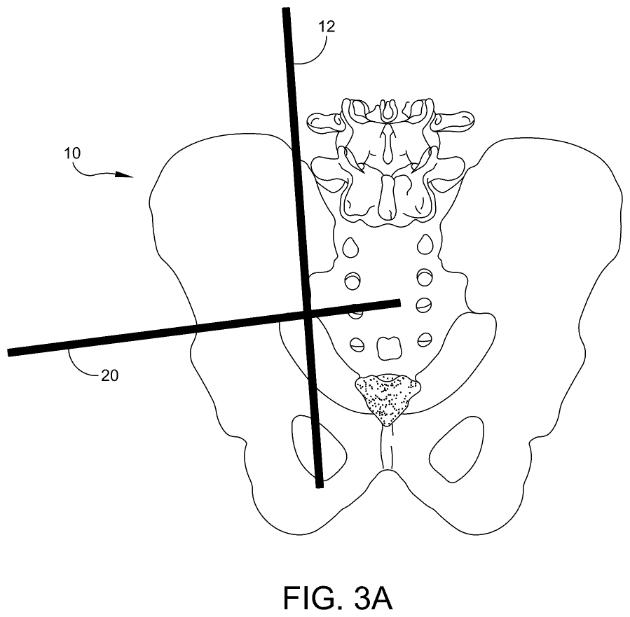

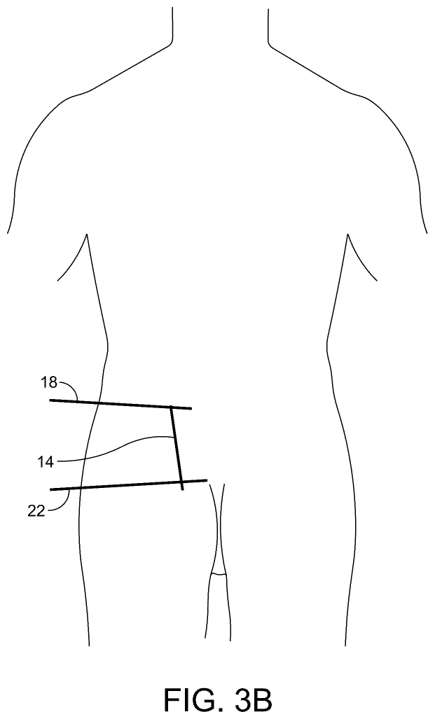

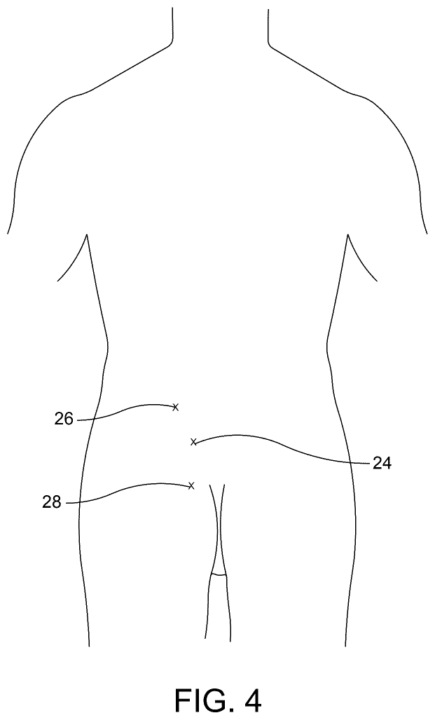

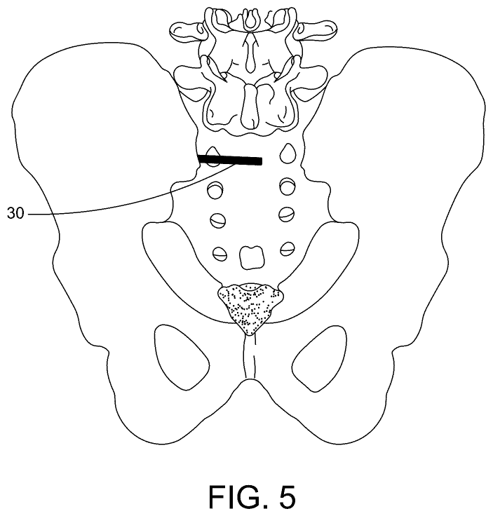

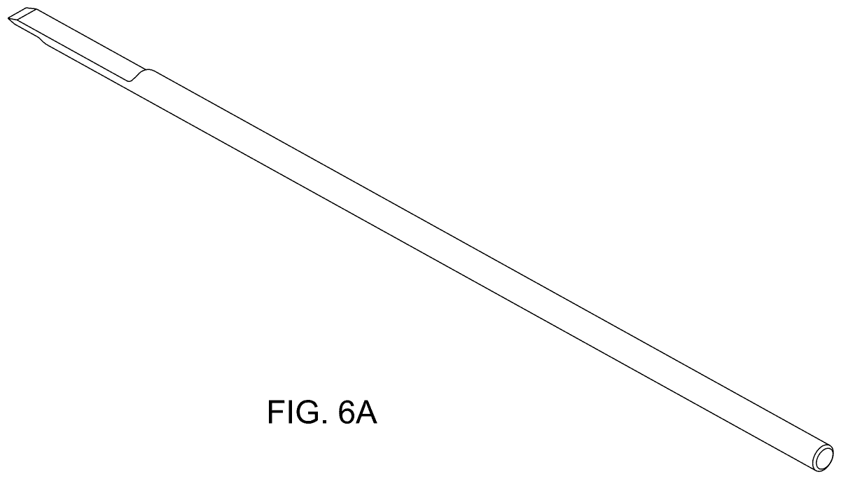

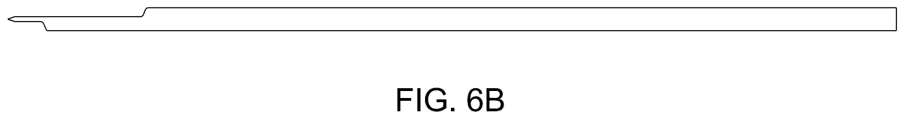

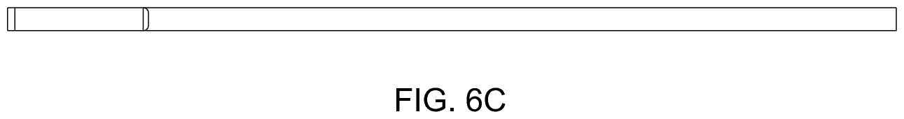

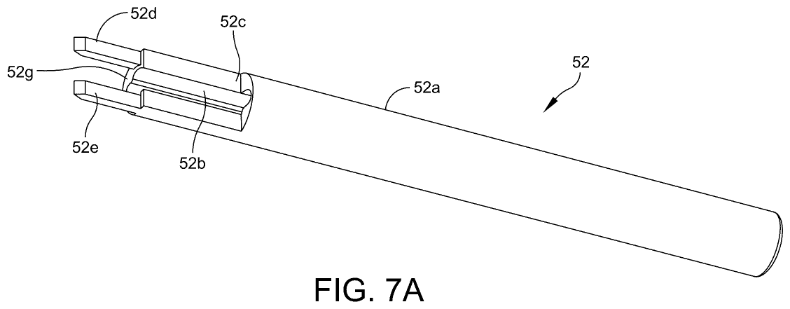

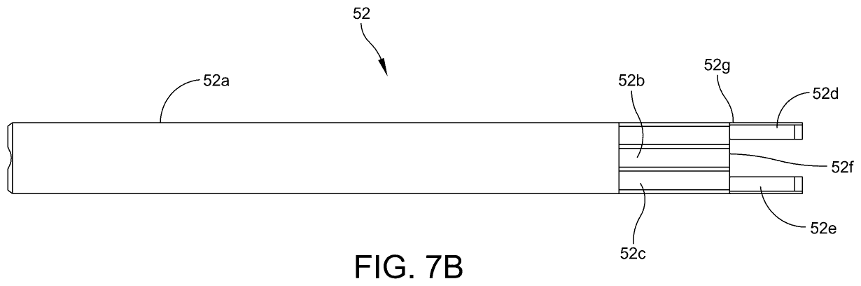

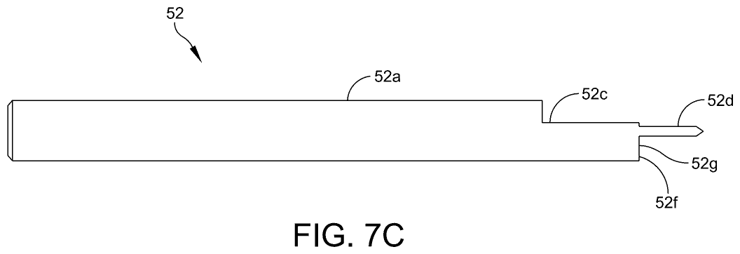

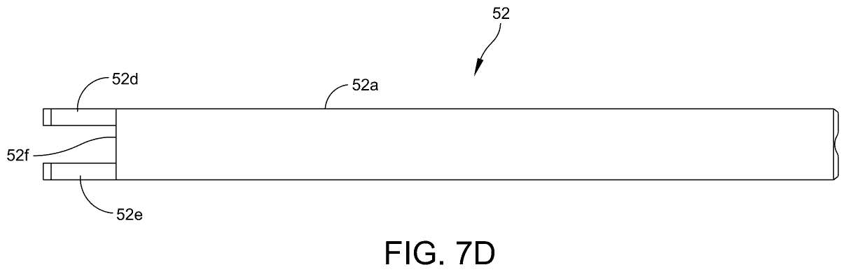

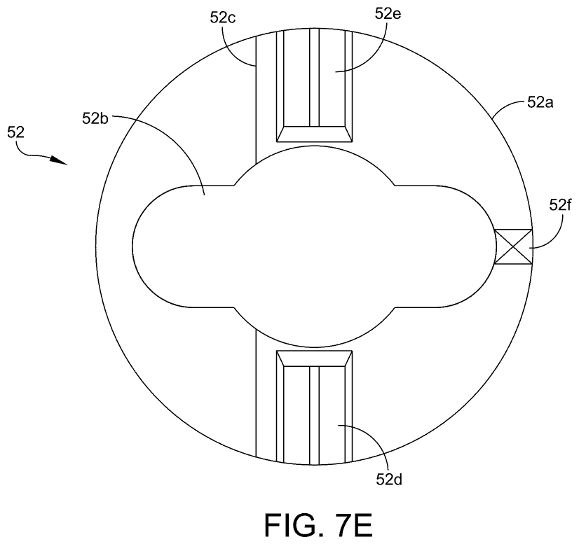

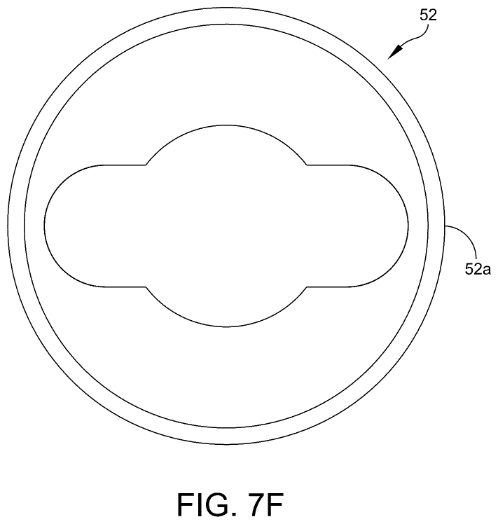

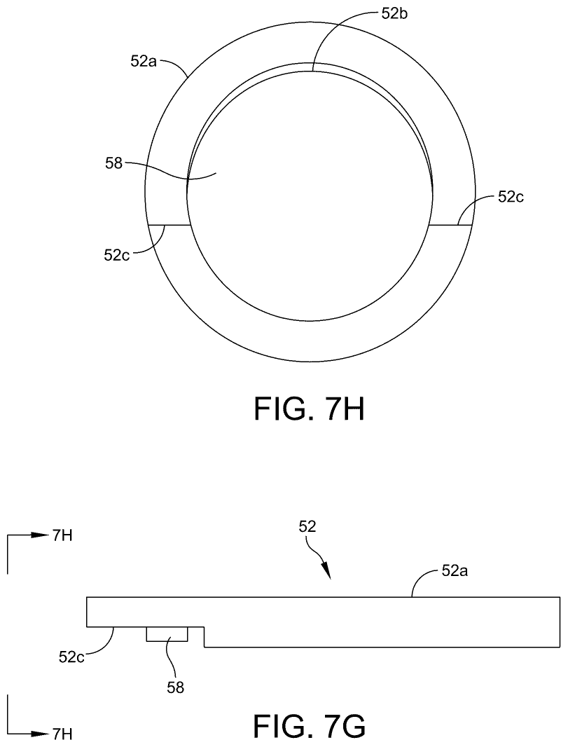

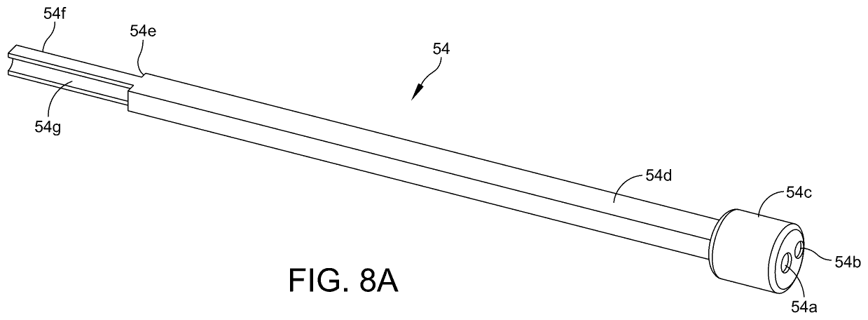

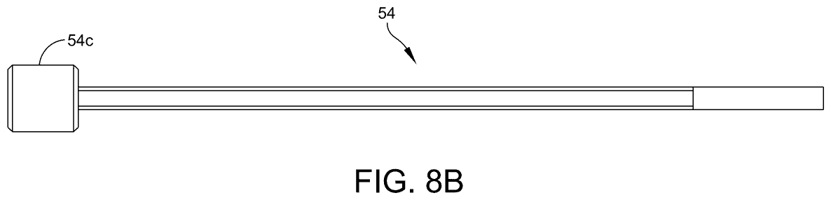

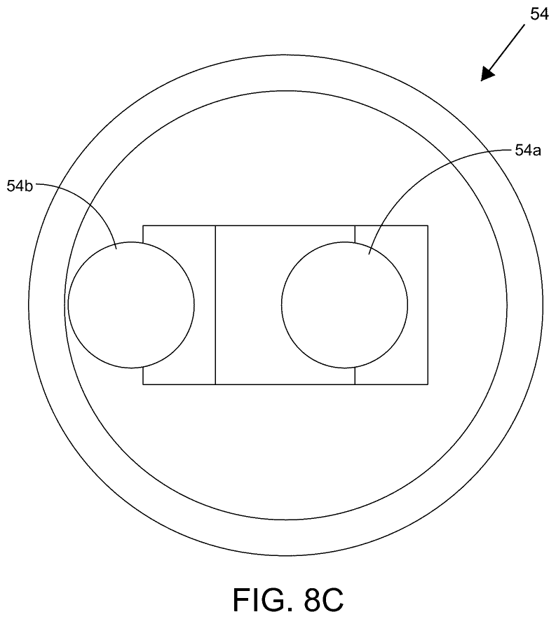

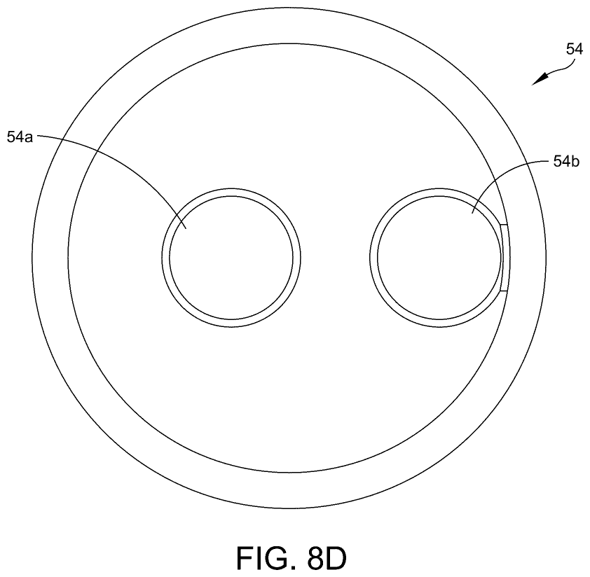

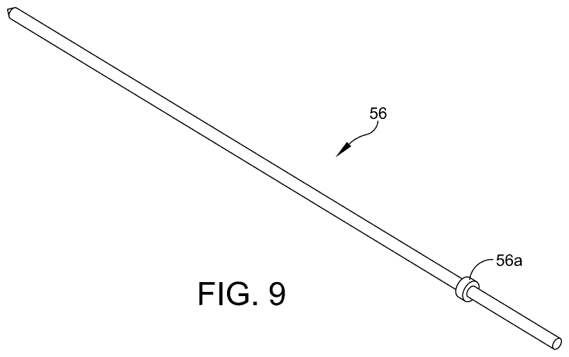

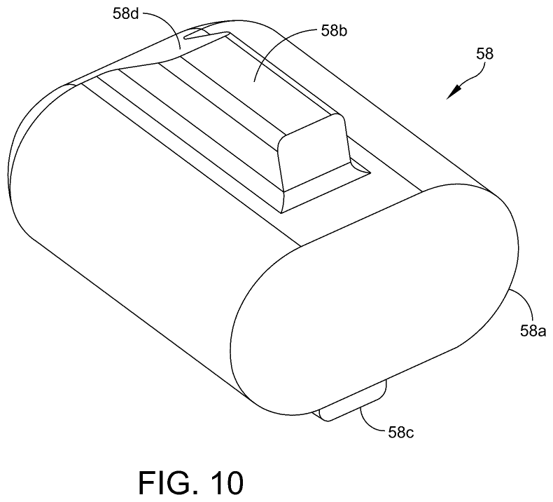

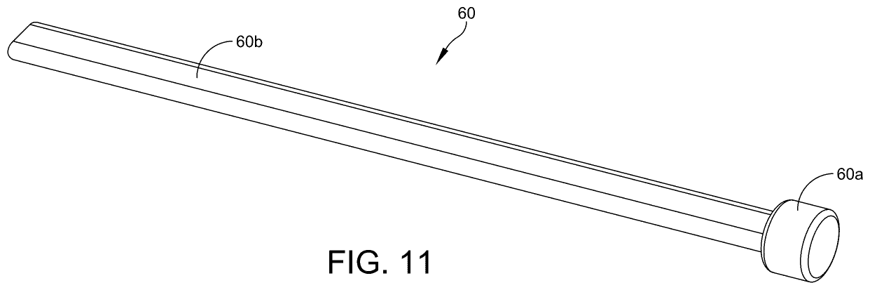

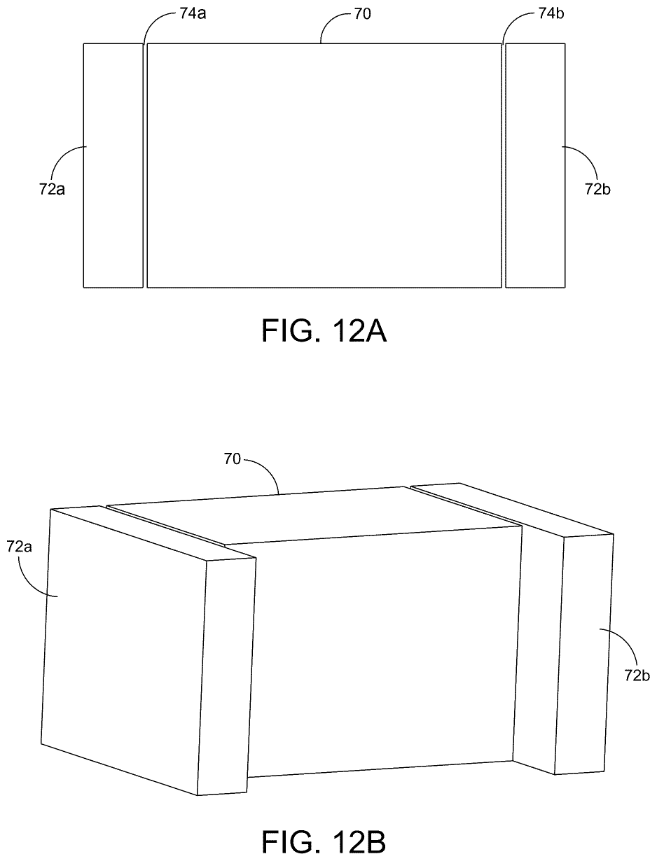

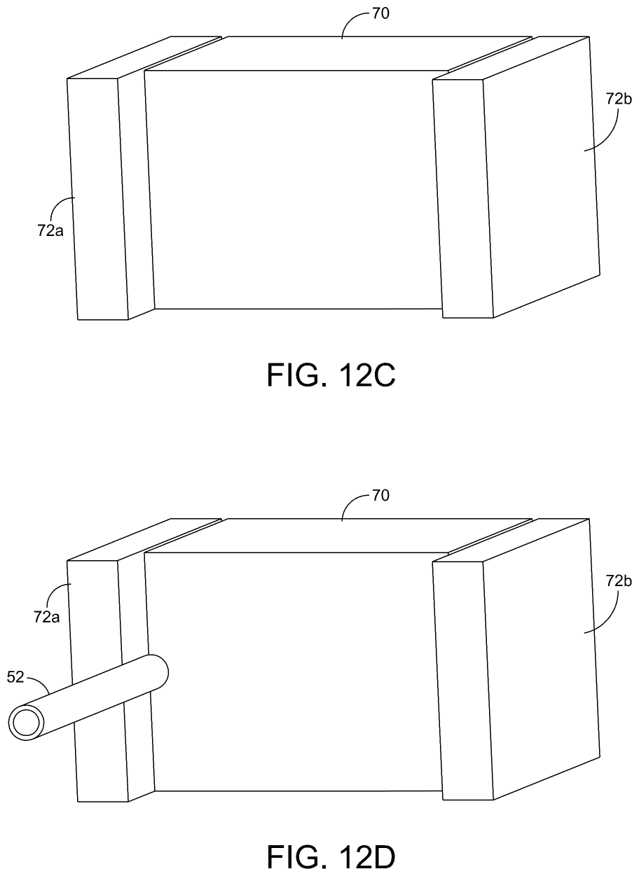

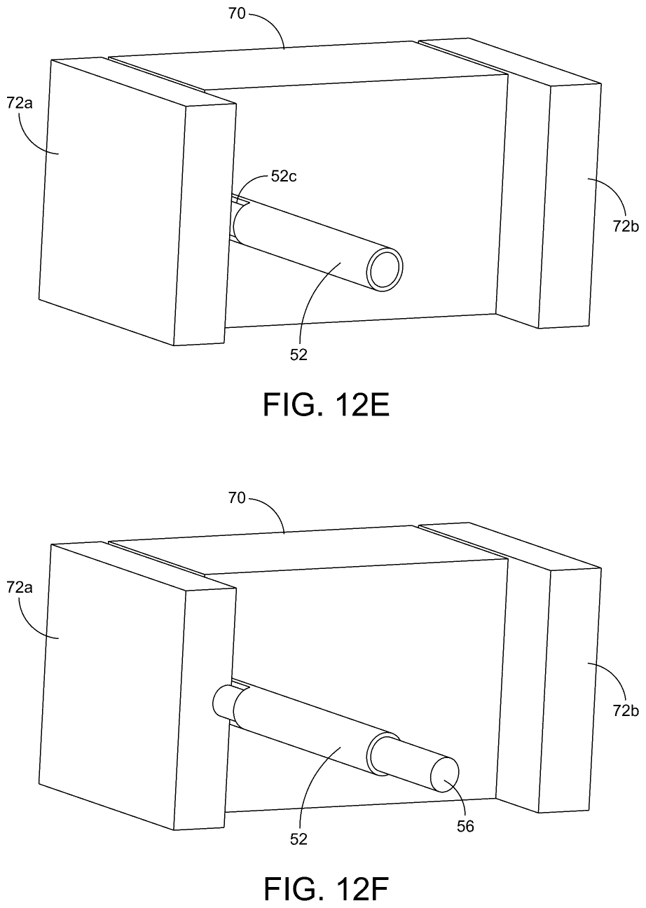

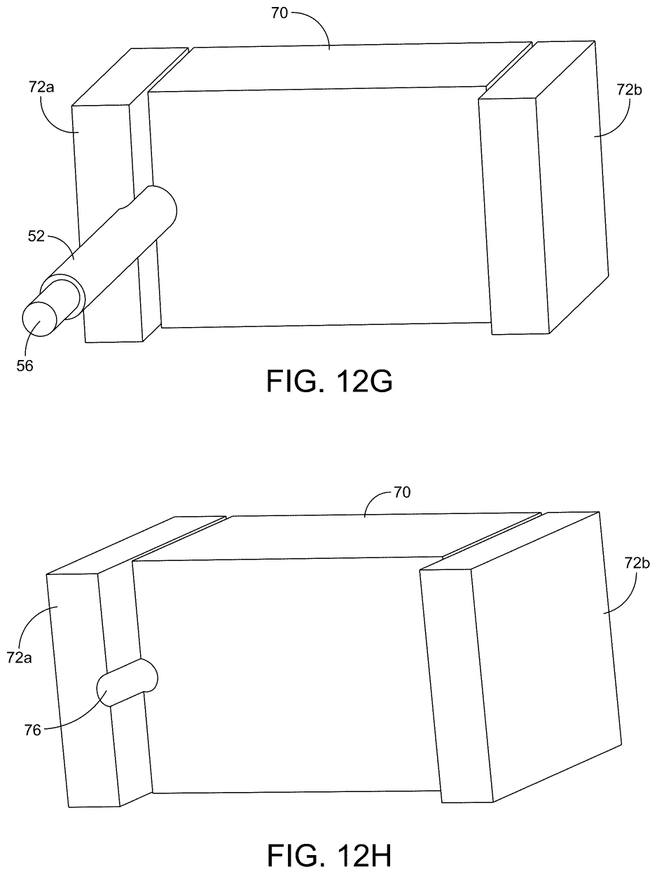

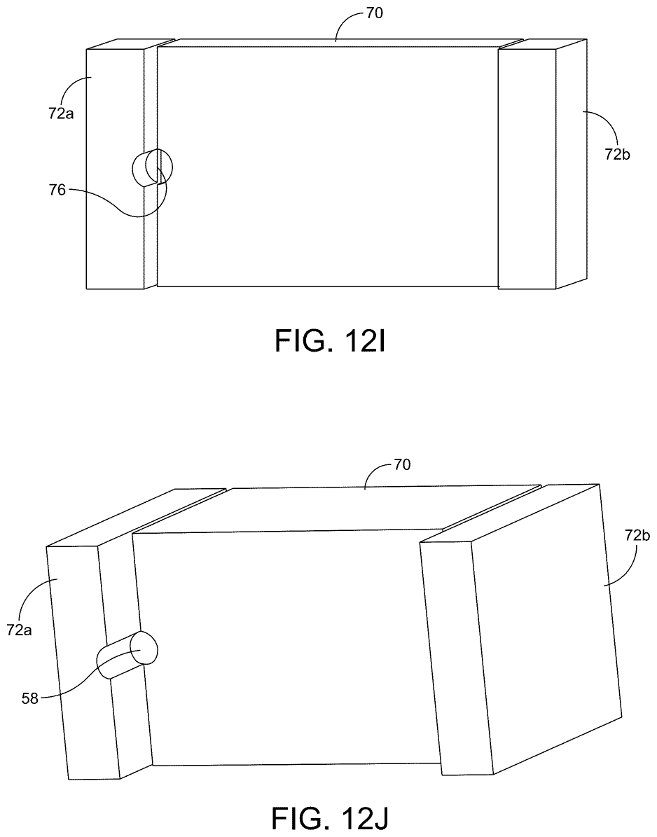

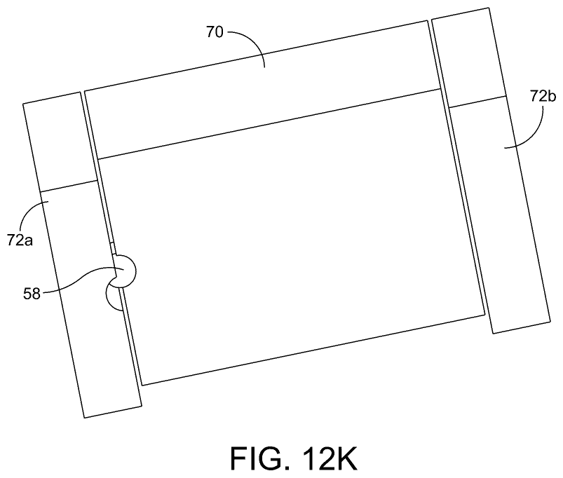

### DETAILED DESCRIPTION O F TH E PREFERRED EMBODIMENTS

### Novel Punch Tool for Forming a Cavity in the Sacroiliac Joint

In the foregoing description, there is described a system comprising a stabilization implant 58 and apparatus for forming a cavity 45 spanning a sacrum 50 and an ilium 55 to receive implant 58. The foregoing apparatus and method for forming cavity 45 generally comprises a joint locator 50, a directional cannula 52, and a drill guide 54 for receiving a drill bit 56. With the foregoing apparatus and method, cavity 45 is generally formed by (i) passing drill guide 54 through directional cannula 52 such that the distal end of the drill guide contacts the joint, (ii) passing drill bit 56 through drill guide 54 and using drill bit 56 to drill a first hole in one of the sacrum or ilium, (iii) removing drill bit 56 and drill guide 54 from directional cannula 52, rotating drill guide 54 (i.e., 180 degrees), and (iv) reinserting drill guide 54 into directional cannula 52, passing drill bit 56 through drill guide 54, and using drill bit 56 to drill a second hole in the other of the sacrum or ilium. The resulting cavity 45 is thus sized to receive stabilization implant 58, which can then be passed through directional cannula 52 and into cavity 45.

However, if desired, in another form of the invention drill guide 54 and drill bit 56 may be omitted, and a novel punch tool system may be used in lieu of drill guide 54 and drill bit 56 in order to form cavity 45.

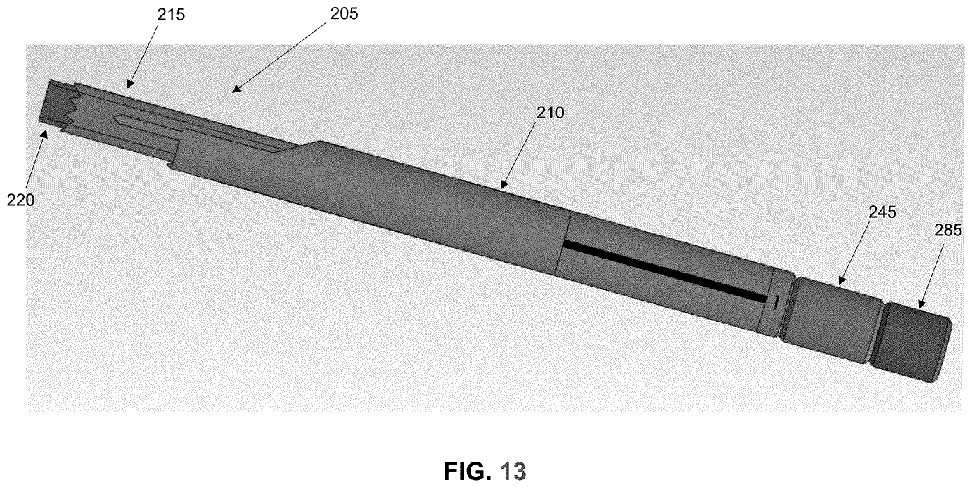

More particularly, and looking now at FIG. 13, there is shown a novel punch tool system 205 disposed in a directional cannula 210.

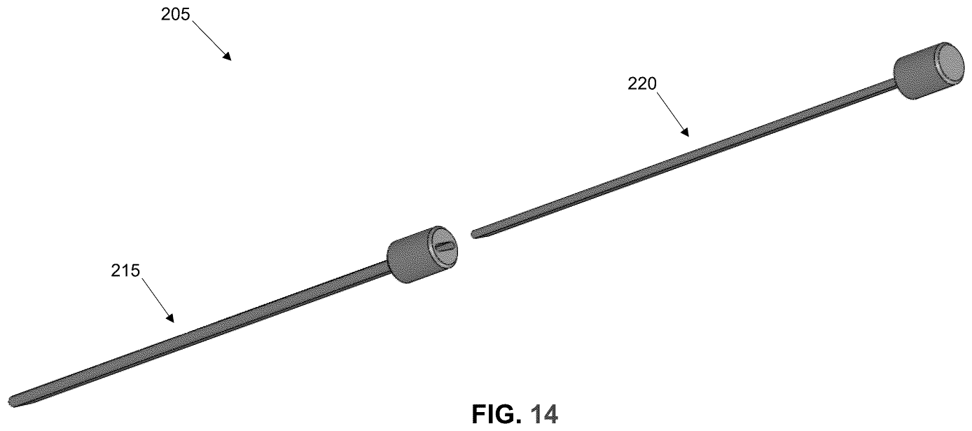

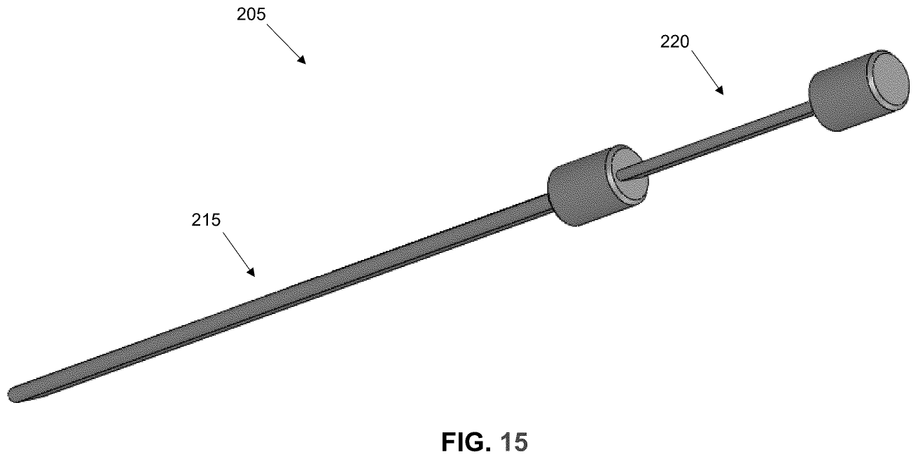

Looking now at FIGS. 14 and 15, punch tool system 205 generally comprises a punch tool guide 215 and a hole punch 220.

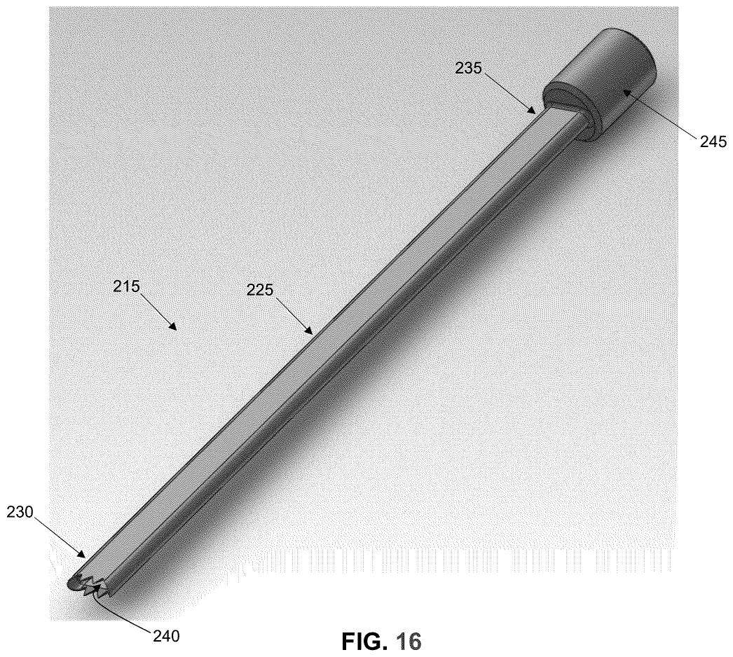

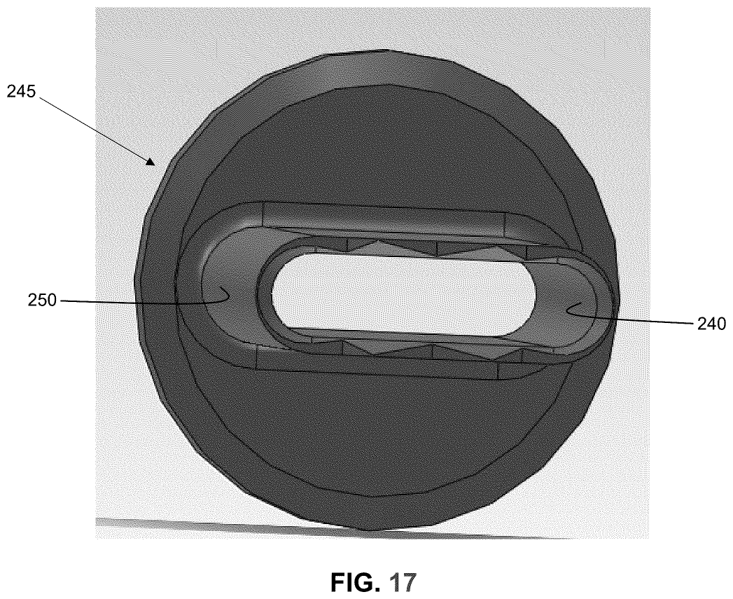

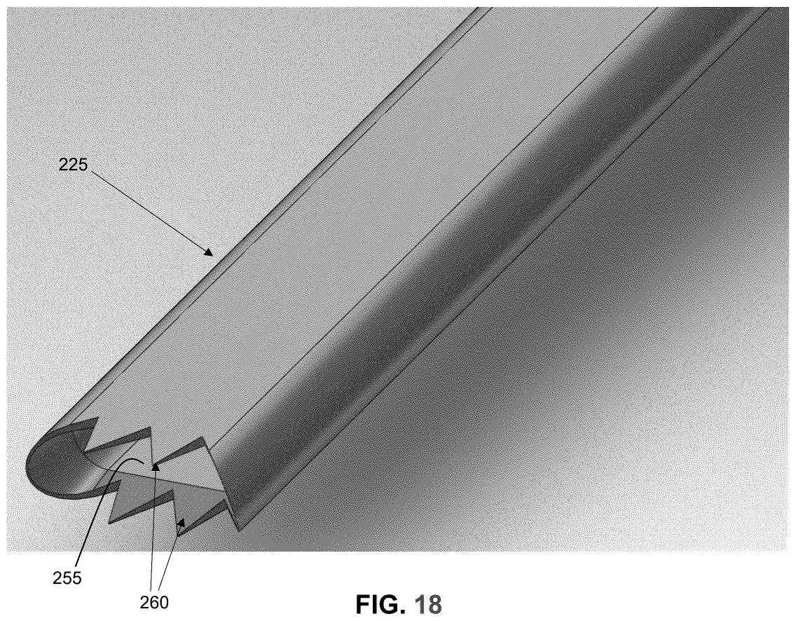

Looking next at FIGS. 16-18, punch tool guide 215 generally comprises a tube 225 having a distal end 230, a proximal end 235, and a lumen 240 extending therebetween. A radially-enlarged stop 245 comprising a lumen 250 (FIG. 17) axially aligned with, and in fluid communication with lumen 240 is mounted to proximal end 235 of tube 225. Distal end 230 of tube 225 terminates in a distal end surface 255. In one preferred form of the invention, distal end surface 255 comprises a plurality of sharpened teeth 260 configured to cut into/grip bone, whereby to facilitate formation of a bone hole using hole punch 220 and/or to assist in gripping the bone tissue so as to maintain punch tool guide 215 in the desired position, as will hereinafter be discussed in further detail.

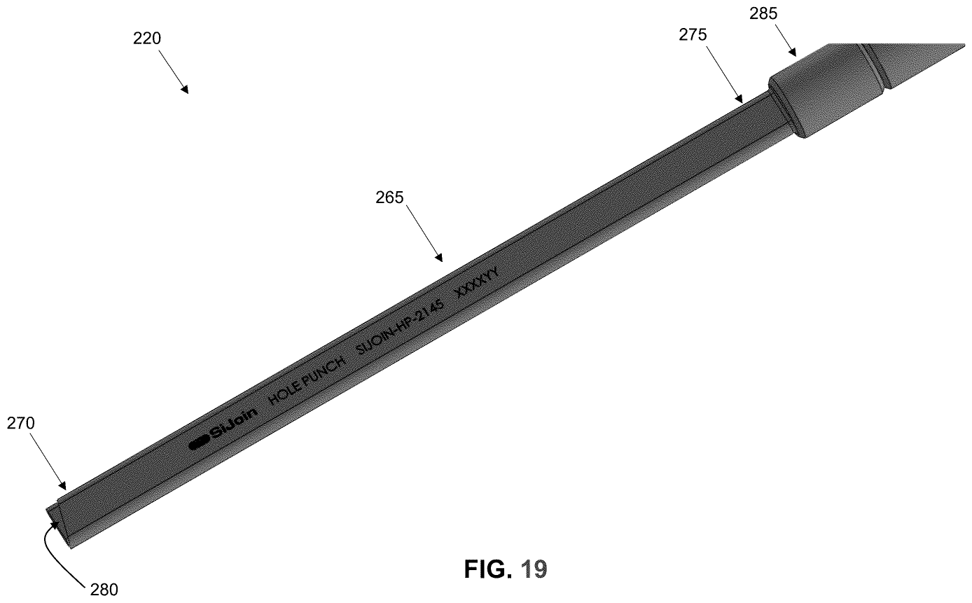

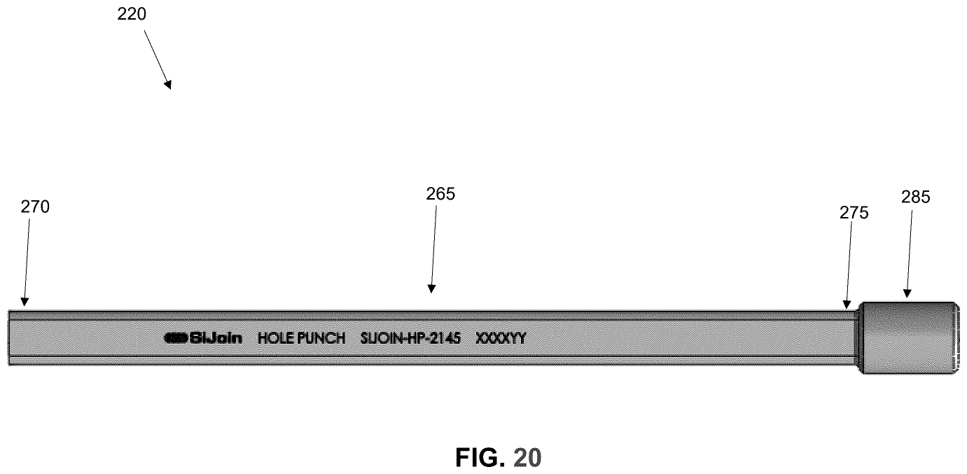

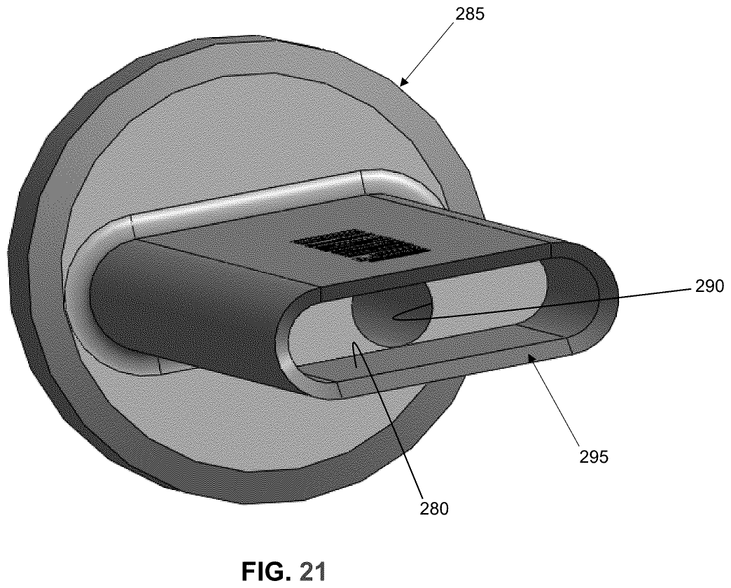

Looking next at FIGS. 19-21, hole punch 220 generally comprises a tube 265 comprising a distal end 270, a proximal end 275, and a lumen 280 extending therebetween. A radially enlarged stop 285 is mounted to proximal end 275 of tube 265. Stop 285 is preferably formed as a solid object that closes off proximal end 275 of tube 265, however, if desired, a central opening 290 axially aligned with lumen 280 of tube 265 may be provided in order to permit a rod (not shown) to be passed through stop 285 and through lumen 280, whereby to be used to eject a cored out plug of bone tissue residing in lumen 280 after use of hole punch 220 to form cavity 45, as will hereinafter be discussed in further detail. It will be appreciated that the cross-section of lumen 280 of tube 265 is configured to match the cross-section of the implant (e.g., the aforementioned stabilization implant 58) that is to be inserted into cavity 45. In a preferred form of the invention, lumen 280 comprises an oblong cross-section. However, it should be appreciated that, if desired, lumen 280 may comprise a different geometry so as to accommodate different geometries of an implant that is to be inserted into cavity 45. By way of example but not limitation, lumen 280 may comprise a circular cross-section, an X-shaped cross-section, a V-shaped cross-section, or a cross-section comprising any other shape/geometry that is needed to match a cross-section of the implant to be inserted into cavity 45.

Distal end 270 of tube 265 terminates in a distal end surface 295. In a preferred form of the invention, distal end surface 295 is formed with a sharpened edge in order to facilitate cutting into bone, as will hereinafter be discussed in further detail.

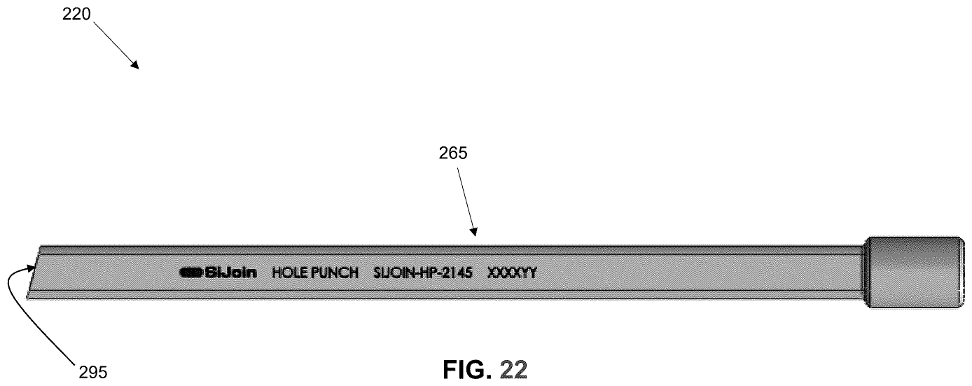

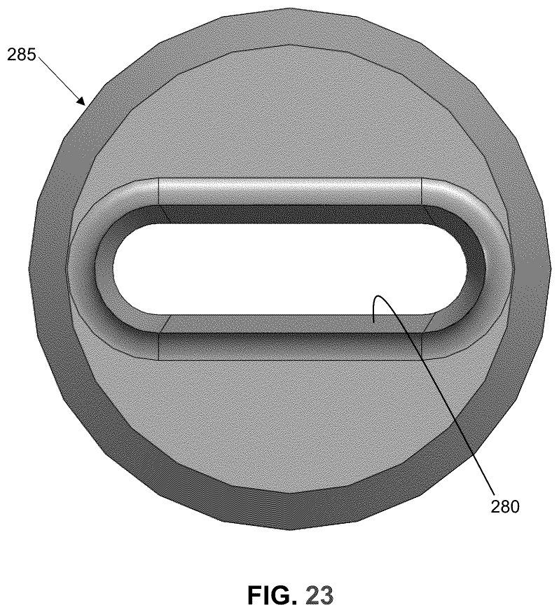

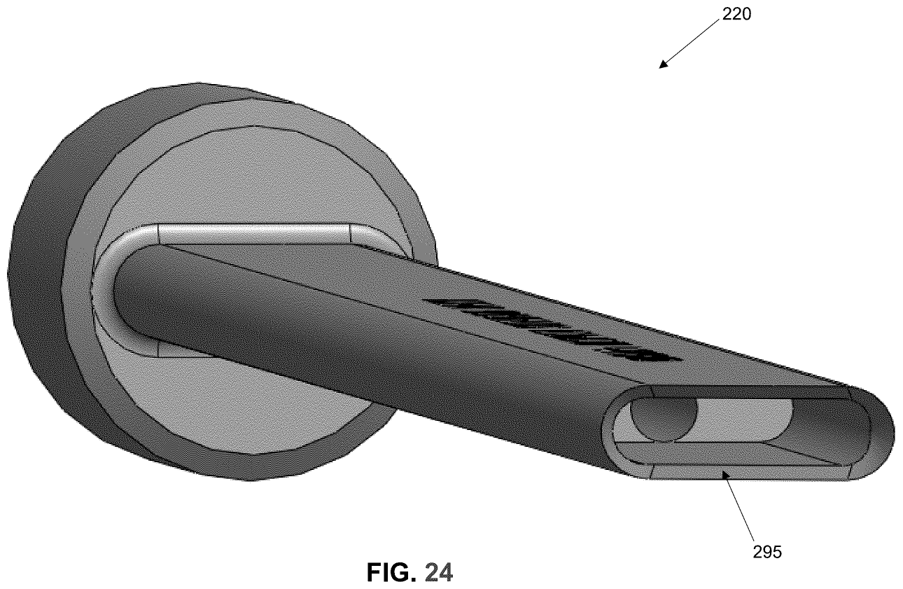

Looking next at FIGS. 22-24, if desired, distal end surface 295 of tube 265 may be formed at an angle relative to the longitudinal axis of tube 265. Alternatively and/or additionally, if desired, distal end surface 295 may be formed with one or more sharpened teeth (not shown) in order to assist in cutting into bone.

In use, the sacrum and ilium are accessed in the same manner as discussed above (i.e., by inserting a joint locator 50 into the joint space, etc.) and directional cannula 210 is inserted into the anatomy such that the distal end of the directional cannula abuts the joint. In a preferred form of the invention, directional cannula 210 comprises a plurality (e.g., two) of distally-extending prongs for insertion into the joint space itself so as to fix the directional cannula in position.

Next, tube 225 of punch tool guide 215 is inserted into directional cannula 210 until stop 245 of punch tool guide 215 contacts the proximal end of directional cannula 210. It will be appreciated that the lengths of directional cannula 210 and of punch tool guide 215 are selected such that punch tool guide 215 extends a predetermined distance beyond the distal end of directional cannula 210. See FIG. 13. In addition, it will also be appreciated that, if desired, punch tool guide 215 may contact the bone of the sacrum and/or ilium before stop 245 engages the proximal end of directional cannula 210 (in which case punch tool guide 215 cannot be moved further distally). Sharpened teeth 260 of punch tool guide 215 preferably cut into the bone tissue and anchor punch tool guide 215 in position.

Next, tube 265 of hole punch 220 is inserted into the proximal end of lumen 250 of punch tool guide 215 and advanced distally until the distal end (i.e., distal end surface 295) of hole punch 220 is disposed distal to distal end surface 255 of punch tool guide 215. It will be appreciated that inasmuch as distal end surface 255 of punch tool guide 215 is coincident with the surface of the bone of the sacrum or ilium, this means that distal end surface 295 of hole punch 220 extends into the bone tissue itself, whereby to core out a volume of bone tissue matching the cross section of lumen 280 of tube 265 of hole punch 220. By controlling the length of tube 265 extending between distal end 270 and stop 285 of hole punch 220, and advancing hole punch 220 distally until stop 285 engages the proximal end of stop 245 of punch tool guide 215, distal end surface 295 extends distally beyond distal end surface 255 of punch tool guide 215 a predetermined distance. Thus, the amount of bone to be cored out is a function of the distance that distal end surface 295 of hole punch 220 extends beyond distal end surface 255 of punch tool guide 215.

It will be appreciated that an important feature of hole punch 220 is that hole punch 220 is configured to create a cavity in hard bone (e.g., the sacrum and the ilium) having a geometry that matches the geometry of an implant to be disposed in the cavity. This is very different than osteotomes, which are used to cut or shave bone, or soft tissue biopsy devices that core out soft tissue. With hole punch 220, it is possible to create a cavity in two adjacent bones over an articulating joint space simultaneously. By way of example but not limitation, hole punch 220 is able to form a portion of such a cavity in a first, medially-disposed bone (e.g., the sacrum) and a portion of such a cavity in a second, laterally-disposed bone (e.g., the ilium, which is disposed lateral to the sacrum). This feature permits a single instrument (i.e., punch tool system 205) to be utilized to form a cavity in two adjacent bones simultaneously, with the cavity having a geometry to match and receive an implant. It will be appreciated that the foregoing features of punch tool system 205 also improve upon systems that require drilling two adjacent bones in order to form a cavity to receive an implant. With such prior art drill-based systems, a drill bit, drill guide and drill are required—all of which is eliminated with punch tool system 205.

Hole punch 220 is then retracted proximally out of lumen 240 of punch tool guide 215, leaving cavity 45 punched out of the sacrum and/or ilium and ready to receive an implant. Punch tool guide 215 may also be retracted proximally out of directional cannula 210, and the implant can be delivered into cavity 45 using directional cannula and/or an implant tamp in a manner that will be apparent to one of skill in the art in view of the present disclosure.

If desired, novel punch tool system 205 may then be used to form additional cavities in the sacrum and/or the ilium for receiving additional implants.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. Apparatus for forming a seat for receiving an implant, the apparatus comprising:
a directional cannula having a distal end, a proximal end, and a cannula lumen extending therebetween;
a punch tool guide having a distal end, a proximal end, and a punch tool guide lumen extending therebetween;
a hole punch comprising a distal end and a proximal end;
wherein the cannula lumen is sized to slidably receive the punch tool guide therein, and the guide lumen is sized to slidably receive the hole punch therein;
wherein when the punch tool guide is disposed in the cannula lumen such that the distal end of the punch tool guide is disposed at a distalmost position, the distal end of the punch tool guide extends distally of the distal end of the directional cannula, and further wherein when the hole punch is disposed in the punch tool guide lumen such that the distal end of the hole punch is disposed at a distalmost position, the distal end of the hole punch extends distally of the distal end of the punch tool guide.

2. Apparatus according to claim 1 wherein the proximal end of the punch tool guide comprises a radially-enlarged guide stop, and further wherein a diameter of the radially-enlarged guide stop is larger than a maximum diameter of the lumen of the directional cannula.

3. Apparatus according to claim 2 wherein the radially-enlarged guide stop comprises a distal surface and a proximal surface, and further wherein the distal surface of the radially-enlarged guide stop engages the proximal end of the directional cannula when the punch tool guide is disposed in the distalmost position.

4. Apparatus according to claim 3 wherein the proximal end of the hole punch comprises a radially-enlarged hole punch stop, and further wherein a diameter of the radially-enlarged hole punch stop is larger than a maximum diameter of the punch tool guide lumen.

5. Apparatus according to claim 4 wherein the radially-enlarged hole punch stop comprises a distal surface and a proximal surface, and further wherein the distal surface of the radially-enlarged hole punch stop engages the proximal surface of the radially-enlarged guide stop when the hole punch is disposed in the distalmost position.

6. Apparatus according to claim 1 wherein the distal end of the directional cannula comprises at least one prong extending distally therefrom.

7. Apparatus according to claim 6 wherein the at least one prong is sized to enter a joint space and stabilize the directional cannula relative to the joint space.

8. Apparatus according to claim 1 wherein the distal end of the punch tool guide comprises a plurality of sharpened teeth.

9. Apparatus according to claim 1 wherein the distal end of the hole punch comprises a sharpened edge for cutting into bone.

10. Apparatus according to claim 1 wherein the lumen of the punch tool guide comprises a non-circular cross-section.

11. Apparatus according to claim 10 wherein the non-circular cross-section of the lumen of the punch tool guide comprises a cross-section matching a cross-section of an implant.

12. A method for forming a cavity in tissue for receiving an implant, the method comprising:
providing apparatus for forming a cavity for receiving an implant, the apparatus comprising:
a directional cannula having a distal end, a proximal end, and a cannula lumen extending therebetween;
a punch tool guide having a distal end, a proximal end, and a punch tool guide lumen extending therebetween; and
a hole punch comprising a distal end and a proximal end;
disposing the distal end of the directional cannula against two bones of a joint;
inserting the punch tool guide into the cannula lumen of the directional cannula and advancing the distal end of the punch tool guide distally towards the joint such that the distal end of the punch tool guide extends distal to the distal end of the directional cannula;
inserting the hole punch into the punch tool guide lumen of the punch tool guide and advancing the distal end of the hole punch distally towards the joint such that the distal end of the hole punch extends distal to the distal end of the directional cannula and the distal end of the punch tool guide; and
forming a cavity in the two bones of the joint.

13. The method according to claim 12 wherein the proximal end of the punch tool guide comprises a radially-enlarged guide stop, and further wherein a diameter of the radially-enlarged guide stop is larger than a maximum diameter of the lumen of the directional cannula.

14. The method according to claim 13 wherein the radially-enlarged guide stop comprises a distal surface and a proximal surface, and further wherein the distal surface of the radially-enlarged guide stop engages the proximal end of the directional cannula when the punch tool guide is disposed in the distalmost position.

15. The method according to claim 14 wherein the proximal end of the hole punch comprises a radially-enlarged hole punch stop, and further wherein a diameter of the radially-enlarged hole punch stop is larger than a maximum diameter of the punch tool guide lumen.

16. The method according to claim 15 wherein the radially-enlarged hole punch stop comprises a distal surface and a proximal surface, and further wherein the distal surface of the radially-enlarged hole punch stop engages the proximal surface of the radially-enlarged guide stop when the hole punch is disposed in the distalmost position.

17. The method according to claim 12 wherein the distal end of the directional cannula comprises at least one prong extending distally therefrom.

18. The method according to claim 17 wherein the at least one prong is sized to enter a joint space and stabilize the directional cannula relative to the joint space.

19. The method according to claim 12 wherein the distal end of the punch tool guide comprises a plurality of sharpened teeth.

20. The method according to claim 12 wherein the distal end of the hole punch comprises a sharpened edge for cutting into bone.

21. The method according to claim 12 wherein the lumen of the punch tool guide comprises a non-circular cross-section.

22. The method according to claim 21 wherein the non-circular cross-section of the lumen of the punch tool guide comprises a cross-section matching a cross-section of an implant.

23. The method according to claim 21 wherein the two bones of the joint are the ilium and the sacrum.
